(19) United States
(12) Patent Application Publication
(10) Pub. No.: US 2008/0305515 A1
(43) Pub. Date: Dec. 11, 2008

(54) Pathology Sample Processing Workstation

(75) Inventors: Lawrence J. Burgart, Rochester, MN (US); Schuyler O. Sanderson, Rochester, MN (US)

Correspondence Address: FAEGRE & BENSON;ATTN: PATENT DOCKETING, 2200 WELLS FARGO CENTER, 90 SOUTH SEVENTH STREET, MINNEAPOLIS, MN, 55402-3901, US

(73) Assignee: Mayo Foundation for Medical Education and Research, Rochester, MN (US)

(21) Appl. No.: 12/063,138

(22) PCT Filed: Aug. 9, 2006
(86) PCT No.: PCT/US06/31012

§ 371 Date: Jun. 16, 2008

Related U.S. Application Data
(60) Provisional application No. 60/706,676, filed on Aug. 9, 2005.

(51) Int. Cl. G01N 1/28; C12M 1/00

(52) U.S. Cl. 435/40.52; 435/283.1

(57) ABSTRACT

A pathology sample processing workstation. The samples are positioned on containers having RFID tags and processed at stations having RFID transceivers coupled to a computer. Information regarding the samples can stored on the RFID tags and retrieved using the transceivers.

### REFERENCE TO RELATED APPLICATION

This application claims the benefit of U.S. Provisional Application Ser. No. 60/706,676 filed on Aug. 9, 2005 and entitled Pathology Sample Processing Workstation, which is hereby incorporated by reference.

### FIELD OF THE INVENTION

The present invention relates generally to workstations and methods for processing pathology tissue samples.

### BACKGROUND OF THE INVENTION

Tissue samples undergoing pathology analyses are typically processed and mounted on slides for viewing using a microscope or other imaging system. The types of processing performed on the samples depends on a variety of factors including the nature of the tissue and the type of analyses to be performed.

There is a continuing need for improved tissue sample processing systems. In particular, there is a need for a system capable of enabling samples to be efficiently and accurately processed for subsequent imaging.

### BRIEF DESCRIPTION OF THE DRAWINGS

FIG. 1 is a schematic illustration of a pathology sample procession workstation in accordance with the present invention.

FIG. 2 is a block diagram of a tissue microtome that can be used in connection with the workstation shown in FIG. 1

FIG. 3 is a block diagram of a microscope that can be used in connection with the workstation shown in FIG. 1.

### DETAILED DESCRIPTION OF THE PREFERRED EMBODIMENTS

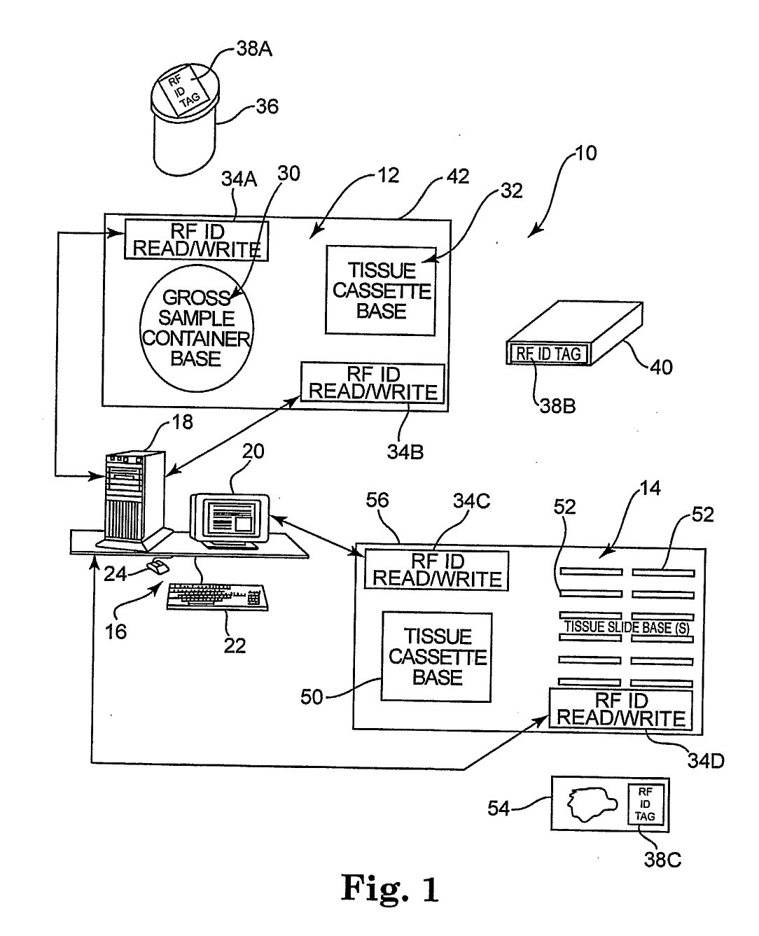

A pathology sample processing workstation 10 in accordance with the present invention is illustrated in FIG. 1. As shown, workstation 10 includes a first work region 12, a second work region 14 and a computer system 16. Computer system 16 can be a commercially available system, and includes a processor 18, monitor 20, keyboard 22 and mouse 24 in the illustrated embodiment. First work region 12 has a first or gross sample container region 30 and a second or cassette sample container region 32. First type containers such as gross sample containers 36 having a radio frequency identification (RFID) tag 38A can be positioned at the first container region 30. Second type containers such as tissue cassettes 40 having RFID tags 38B can be positioned at the second container region 32. RFID transmitters and/or receivers such as read/write transceivers 34A and 34B are located in operative communication with the RFID tags 38A and 38B of the containers 36 and 38, respectively, when the containers are positioned at the container regions 30 and 32. RFID read/write transponders 34A and 34B are operatively coupled to computer system 16.

In the embodiment shown, the gross sample containers 36 have a base with a first shape or other configuration, and the tissue cassettes 40 have a base with a second shape or other configuration that is different from that of the gross sample containers. The first container region 30 can have an indicia, raised lip or other feature that corresponds (e.g., is complimentary) to the first configuration of the sample containers 36 so as to facilitate the positioning, orientation and/or registration of the container and its RFID tag 38A with respect to the associated read/write transponder 34A. In the embodiment shown, container regions 30 and 32 and the RFID transponders 34A and 34B are incorporated into a polymer base 42 that can be positioned on a lab bench or other work station. The polymer base protects the components of the first work region 12 from fluids and/or chemicals associated with tissue processing. Alternatively, the work region 12 can be a region on a larger work area such as a lab bench.

Similarly, second work region 14 has a third or cassette sample container region 50 and a fourth or slide sample container regions 52. The cassette sampler container region 50 can be the same as or similar to the cassette sample container region 32 of work region 12 described above. Containers such as tissue cassettes 40 having RFID tags 38B can be positioned at the third sample container region 50. Third type containers such as sample slides 54 having RFID tags 38C can be positioned at the fourth container regions 52. RFID transmitters and/or receivers such as read/write transceivers 34C and 34D are located in operative communication with the RFID tags 38B and 38C of the containers 40 and 54, respectively, when the containers are positioned at the container regions 50 and 52. RFID read/write transceivers 34C and 34D are operatively coupled to computer system 16.

In the embodiment shown, the third container region 50 has an indicia, raised lip or other feature that corresponds (e.g., is complimentary) to the configuration of the tissue cassette 40 so as to facilitate the positioning, orientation and/or registration of the tissue cassette and its RFID tag 38B with respect to the associated read/write transceiver 34C. Similarly, sample slides 54 have a shape or other configuration that is different than that of the tissue cassette 40, and the fourth container regions 52 have indicia, raised lips or other features that correspond to the configuration of the sample slides. These features facilitate the positioning, orientation and/or registration of the sample slides 54 and their RFID tags 38C with respect to the associated RFID read/write transceiver 34D. In the embodiment shown, container regions 50 and 52 and the RFID transponders 34C and 34D are incorporated into a polymer base 56 that can be positioned on a lab bench or other work station. The polymer base protects the components of the second work region 14 from fluids and/or chemical associated with tissue processing. Alternatively, the work region 14 can be a region on a larger work area such as a lab bench.

In operation, a container 36 having a gross sample to be processed can be positioned at container region 30. Computer system 16 can then be used to read and/or record sample information on the RFID tag 38A of the sample containers 36. Examples of the types of sample information that can be transferred to or from the tag 38A include patient name or other source identification, sample type, requested analyses and requested processing. Other types of information that can be transferred to or from the RFID tag 38A include, the stain, antibody, fixative, etc. that is to be performed on the sample based on the type of tissue, type of disease, or type of information a physician wanted assessed. Information stored in computer system 16 can be transferred to the RFID tag 38A as part of this operation.

A clinician can process the sample (not shown) removed from container 36, and place the processed sample in a tissue cassette 40 located at container region 32. The specific processing steps that are performed can be done as a function of the information read from the RFID tag 38. Through use of the computer system 16 and RFID read/write transceiver 34B, the clinician can transfer some or all of the sample information from RFID tag 38A to RFID tag 38B associated with the processed sample. Similarly, additional sample information can be added to the RFID tag 38B of the tissue cassette 40 in which the processed sample is located. Examples of the additional sample information include information on the processing that was performed and the results of that processing.

Following the steps described above, the tissue cassette 40 can be transferred to and positioned on the container region 50 of workstation 14. Tissue samples can then be removed from the cassette 40 and mounted on slides 54 located at container regions 52. In connection with these sample mounting operations, sample information can be transferred to or from the RFID tag 38B of the cassette 40 and the RFID tag 38C of the slide 54 to which the sample is mounted. Computer system 16 can be operated in connection with RFID read/write transceivers 34C and 34D to provide this information transfer. Again, both sample information read from the RFID tag 38B of the tissue cassette 40 and/or information regarding the associated tissue stored on computer system 16 can be transferred to the RFID tag 38C on the slide 54. Alternatively, or in addition, additional processing information can be transferred to the RFID tags 38C of individual slides 54 or groups of slides. In this manner a given slide 54 can be programmed and subsequently interrogated for both sample identity and to determine the processing steps that were or are to be performed on the sample.

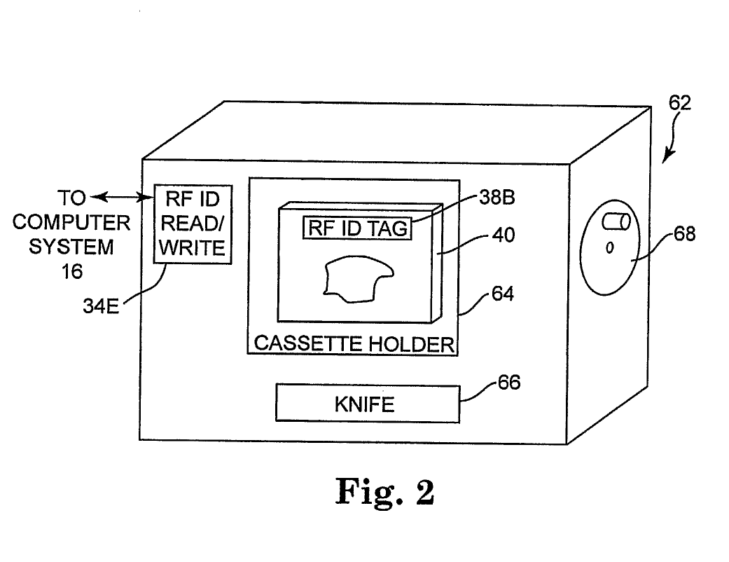

FIG. 2 is an illustration of a tissue microtome 62 that can be used in connection with workstation 10 to created sample slides 54 from tissue cassettes 40. As shown, microtome 62 includes a cassette holder 64, knife 66 and actuator 68. Cassette holder 64, knife 66 and actuator 68 can be components of commercially available microtomes. Microtome 62 also includes an RFID read/write transceiver 34E that is operatively coupled to computer system 16. Transceiver 34E is located on the microtome 62 at a position that enables operative communication with the RFID tags 38B of tissue cassettes being processed by the microtome. In other embodiments of the invention (not shown) the transceiver 34E can be positioned at locations off the microtome 62, yet still be capable of communications with the RFID tags 38B of tissue cassettes 40 being processed by the microtome.

Sample slides 54 can be created using the microtome 62. Histotechs preparing the slides can place cassette 40 on the cassette holder 64 of the microtome 62. The sample is typically embedded on the cassette 40 by paraffin. Actuator 68 is operated to cause the holder 64 to move the cassette 40 and the tissue thereon over the knife 66 to cut microscopic sections. These sections are then placed onto slides 54 and labeled with appropriate case identification and other information. Through use of the RFID tag 38B on the cassette 40 and transceiver 34E on the microtome 62, maintenance of the identity integrity and other relevant information can be ensured during the creation of the sample slide 54.

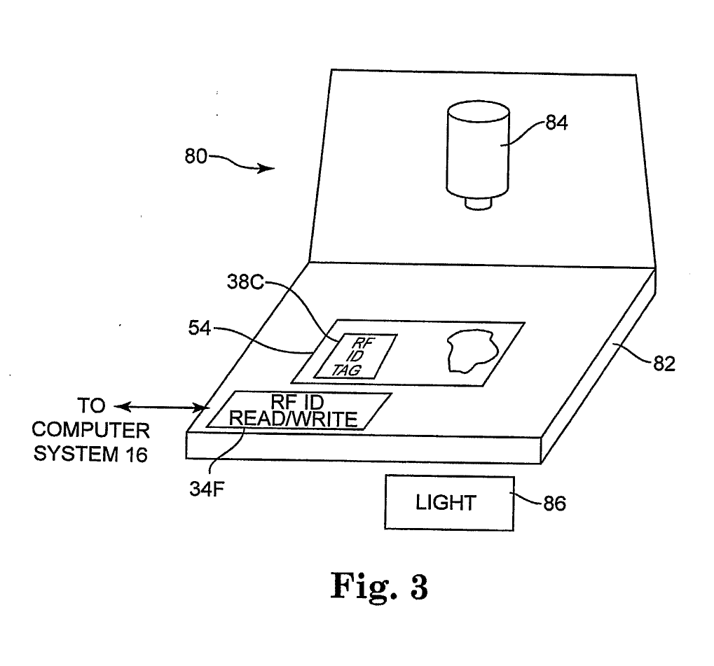

FIG. 3 is an illustration of a microscope 80 that can be used in connection with workstation 10 to view sample slides 54. As shown, microscope 80 includes a stage 82 located between multiple magnification objectives 84 and a light source 86. Stage 82, objectives 84 and light source 86 can be components of commercially available microscopes. Microscope 80 also includes an RFID read/write transceiver 34F that is operatively coupled to computer system 16. Transceiver 34F is located on the microscope 80 at a position that enables operative communication with the RFID tags 38C of sample slides 54 being evaluated using the microscope. In the illustrated embodiment, for example, the transceiver 34F is located on stage 82. In other embodiments of the invention (not shown) the transceiver 34F can be positioned at locations off the microscope 80 yet still be capable of communications with the RFID tags 38C of sample slides 54 on the microscope.

During the diagnostic evaluation of sample slides 54 a pathologist can position the slides on the stage 82. Information contained on the RFID tags 38C on the slides 54 can be read by the transceiver 34F and used for any of a variety of purposes including identification integrity in connection with the creation of the diagnostic report. Computer system 16 communicates with the transceiver 34F to created a link between the slides 54 for a particular case and an electronic diagnostic report that can be relayed back to the patient's clinician.

The invention offers important advantages. In particular, it enables samples to be efficiently and accurately processed for subsequent processing. A clinician using the computer system can also track a particular sample in the preparation process and verify that the desired sample processing has occurred. Commercially available RFID systems and components can be used.

Although the present invention has been described with reference to preferred embodiments, those skilled in the art will recognize that changes can be made in form and detail without departing from the spirit and scope of the invention. For example, although an RFID read/write transceiver is shown associated with each container region, fewer transceivers can be used. For example, fewer RFID transceivers can be used in application where the transceivers can discriminate between RFID tags at different locations.

What is claimed is:

1. A pathology sample processing system including:
a plurality of container regions for receiving sample containers having information storage and retrieval devices; and
read and/or write transceivers associated with at least some of the container regions, for reading and/or writing sample information to the storage and retrieval devices on containers positioned at the container regions.

2. The pathology sample processing system of claim 1 and further including a computer system coupled to the read and/or write transceivers.

3. The pathology sample processing system of claims 2 wherein the container regions include indicia, raised lips or other features that correspond to the configuration the sample containers, to facilitate the positioning, orientation and/or registration of sample containers at the container regions.

4. The pathology sample processing system of claim 3 wherein at least two of the indicia, raised lips or other features that correspond to the configuration the sample containers are different than one another.

5. The pathology sample processing system of claim 4 wherein the system includes:
a first workstation having a first and second container regions, and wherein the first and second container regions are different than one another; and
a second workstation having third and fourth container regions, and wherein the third container region is the same as the second container region on the first workstation.

6. The pathology sample processing system of claim 5 wherein the fourth container region is a slide region.

7. The pathology sample processing system of claim 1 and further including one or more polymer bases to which at least some of the container regions and associated read/write transceivers are mounted.

8. The pathology sample processing system of claim 1 and further including a microtome having a read and/or write transceiver for reading and/or writing information to storage and retrieval devices on containers located on the microtome.

9. The pathology sample processing system of claim 1 and further including a microscope having a read and/or write transceiver for reading and/or writing information to storage and retrieval devices on containers located on the microscope.

10. The pathology sample processing system of claim 1 wherein:
the information storage and retrieval devices are RFID tags; and
the read and/or write transceivers are RFID transceivers.

11. A method for using a pathology sample processing system, including:
positioning one or more sample containers having information storage and retrieval devices thereon on container regions having read and/or write transceivers;
processing a sample in one or more of the containers; and
operating a computer system to transfer sample information to or from the information storage and retrieval devices on sample containers through use of the read and/or write transceivers.

12. The method of claim 11 with sample containers having different configurations, including:
positioning, orienting or registering one or more sample containers on container regions of the system having indicia, raised lips or other features that correspond to the configuration the sample containers;
processing a sample in one or more of the containers; and
operating the computer system to transfer sample information to or from the information storage and retrieval devices on sample containers through use of the read and/or write transceivers.

13. The method of claim 12 wherein operating the computer system to transfer sample information includes transferring sample information of the type including patient name or other source identification, sample type, requested analyses, requested processing, the stain, antibody or fixative that is to be performed on the sample, information on the processing that was performed on the sample, and the results of sample processing.
